(19) United States
(12) Patent Application Publication
(10) Pub. No.: US 2016/0158309 A1
(43) Pub. Date: Jun. 9, 2016

(54) EXERCISE FUNCTION ENHANCER

(71) Applicants: KOHJIN LIFE SCIENCES CO., LTD., Tokyo (JP); KYOTO PREFECTURAL PUBLIC UNIVERSITY CORPORATION, Kyoto (JP)

(72) Inventors: Wataru AOI, Kyoto (JP); Toru KONISHI, Saitama (JP); Yusuke SAUCHI, Tokyo (JP)

(73) Assignees: KYOTO PREFECTURAL PUBLIC UNIVERSITY CORPORATION, Kyoto (JP); KOHJIN LIFE SCIENCES CO., LTD., Tokyo (JP)

(21) Appl. No.: 14/966,050

(22) Filed: Dec. 11, 2015

Related U.S. Application Data
(63) Continuation of application No. 14/563,213, filed on Dec. 8, 2014.

(51) Int. Cl. A61K 38/06; A61K 9/00

(52) U.S. Cl. CPC A61K 38/063; A61K 9/0053

(57) ABSTRACT

The inventors of the present invention have discovered that glutathione inhibits reduction in muscle pH, and activates PGC-1α to increase production of mitochondrial DNA. The present invention has been completed based on these findings.

### CROSS-REFERENCE TO RELATED APPLICATION

This application is a Continuation of U.S. application Ser. No. 14/563,213, filed on Dec. 8, 2014. The disclosure of application Ser. No. 14/563,213 is expressly incorporated by reference herein in their entireties.

### BACKGROUND OF THE INVENTION

1. Field of the Invention

The present invention relates to an exercise function enhancer containing glutathione as an active component.

2. Description of Related Art

Typically, when exercising, muscle glycogen-derived glucose is utilized, and an anaerobic glycolytic reaction develops to synthesize ATP, which is necessary for muscle contraction. Due to accumulation of lactic acid, which is a metabolic product of the above, pH in the muscles is reduced and efficiency of muscle contraction drops off. Therefore, the reduction of pH in the muscles must be prevented in order to maintain exercise function.

PGC-1α is a known factor contributing to regulating energy metabolism in skeletal muscle (Cell, 92, 829-838, 1998). When mice are placed in a cold environment, PGC-1α in the skeletal muscles increases. Therefore, PGC-1α is known to be involved in regulating thermogenesis in skeletal muscle tissue. In addition, forced expression of PGC-1α induces expression of NRF (promoting transcription of factors involved in mitochondrial respiratory chains) and uncoupling proteins (UCP, believed to cause energy consumption in mitochondria), as well as expression of mitochondrial transcription factor A (mtTFA, which plays an important role in mitochondrial genome replication and transcription reaction processes). It is clear that, due to the expression of functions of these molecules, the number of mitochondria within a cell increased, as did the oxygen consumption of the cell. Therefore, it is known that by activating mitochondrial function in human-derived cells, thermogenesis (i.e., energy consumption) is induced, and metabolism of sugars and lipids (energy sources within the cell) is activated (Cell, 98, 115-124, 1999).

To this point, exercise function enhancers such as anti-fatigue agents have been known, examples of which include vitamins (Japanese Patent Laid-open Publication No. 2010-138170), imidazole compounds contained in large amounts in bonito or tuna (Japanese Patent Laid-open Publication No. 2002-338473), and ornithine (World Intellectual Property Publication No. WO2007/142286). Glutathione, a known antioxidant, is described in World Intellectual Property Publication No. WO/2007/142286 as having a stress-related anti-fatigue effect due to a synergistic effect with orthinine.

However, to this point glutathione has not been known to maintain or improve exercise function.

### SUMMARY OF THE INVENTION

The present invention provides an exercise function enhancer that maintains and improves exercise function using a safe substance.

The inventors of the present invention have discovered that glutathione inhibits reduction in muscle pH, and activates PGC-α to increase production of mitochondrial DNA. The present invention has been completed based on these findings.

The present invention provides:

(1) an exercise function enhancer containing glutathione as an active component and enhancing exercise function due to mitochondrial activity (increase in an amount of mitochondrial DNA);
(2) an exercise function enhancer containing glutathione as an active component and enhancing exercise function due to inhibiting reduction in muscle pH;
(3) an exercise function enhancer containing glutathione as an active component and enhancing exercise function due to mitochondrial activity (increase in an amount of mitochondrial DNA) and inhibiting reduction in muscle pH.

According to the present invention, exercise function can be maintained due to the inhibition of reduction in muscle pH. In addition, because mitochondria are activated by the activation of PGC-1α, uptake and burning of lipids in the body proceeds efficiently, and therefore not only is exercise function maintained, but lipid metabolism in the body is also promoted. Therefore, lifestyle diseases such as diabetes may be prevented.

### BRIEF DESCRIPTION OF THE DRAWINGS

The present invention is further described in the detailed description which follows, in reference to the noted plurality of drawings by way of non-limiting examples of exemplary embodiments of the present invention, in which like reference numerals represent similar parts throughout the several views of the drawings, and wherein:

FIG. 1 illustrates pH in skeletal muscle (glutathione);

FIG. 2 illustrates pH in skeletal muscle (vitamin C);

FIG. 3 illustrates PGC-1α expression in skeletal muscle (glutathione);

FIG. 4 illustrates PGC-1α expression in skeletal muscle (vitamin C); and

FIG. 5 illustrates an amount of mitochondrial DNA in skeletal muscle (glutathione).

### DETAILED DESCRIPTION OF THE INVENTION

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show structural details of the present invention in more detail than is necessary for the fundamental understanding of the present invention, the description taken with the drawings making apparent to those skilled in the art how the forms of the present invention may be embodied in practice.

In the present invention, “improvement in exercise function” refers not to inhibiting fatigue, but rather to improving effectiveness in various kinds of exercise, from exercise during competition to light exercise such as ordinary jogging and walking. The present invention also encompasses promoting lipid metabolism within the body not only while exercising, but also during daily activities such as walking and other normal activities.

According to the present invention, inhibiting reduction of pH in muscle tissue generally involves reducing and acidifying pH in muscles due to an accumulation of lactate in muscle tissue. When the pH in muscle tissue is reduced, metabolic activity of the muscle tissue is reduced and exercise cannot be sustained. The present invention inhibits the reduction of pH in muscle tissue; therefore, exercise can be maintained. Moreover, because the present invention has a similar effect not only during exercise, but also in daily activities such as walking, consumption of glucose, for example, during daily activities is increased.

PGC-1α refers to “peroxisome proliferator-activated receptor γ co-activator 1α,” and is known to promote mitochondrial synthesis and to increase GLUT4, which is a glucose transporter delivering glucose in the blood (blood sugar) to skeletal muscle. Further, PGC-1α expression in human muscle is reduced, along with mitochondrial function, by diabetes and old age, and PGC-1α is a known target as a treatment for lifestyle diseases such as metabolic syndromes caused by a decrease in energy consumption.

By supplementing PGC-1α and the inhibition of reduction in muscle tissue pH, the present invention increases the amount of mitochondrial DNA and effectively promotes mitochondrial synthesis. Therefore, the present invention can also be employed to prevent lifestyle diseases.

In the present invention, glutathione refers to reduced glutathione, oxidized glutathione, or a mixture of the same. “Reduced glutathione” means a tripeptide having the structure γ-L-Glu-L-Cys-Gly, while “oxidized glutathione” has two molecules of reduced glutathione joined by an SS bond. The glutathione may have any form so long as it contains glutathione as an active component.

In order to achieve the improved exercise function of the present invention, a substance containing the above-noted glutathione as an active component is administered. Administration methods, while not particularly limited, can include oral administration or non-oral administration, such as intravenous, peritoneal, or dermal administration. Specifically, the present invention may be administered by either an oral medication, such as a tablet, powder, granule, pill, suspension, emulsion, infusion/decoction, capsule, syrup, liquid, elixir, extract, tincture, or fluid extract; or a non-oral medication, such as an injection, drops, cream, or suppository.

Oral administration of yeast containing glutathione is also possible. An example of a yeast with high glutathione content is “HITHION MG” (manufactured by Kohjin Life Sciences Co., Ltd.), while an example of a yeast extract containing glutathione is “HITHION extract YH” (manufactured by Kohjin Life Sciences Co., Ltd.).

A dosage of the present invention is not particularly limited so long as the above-described functions are achieved. When administering to humans, a number of doses and a dosage amount vary according to the form of administration and a patient's age, weight, and the like. However, a typical adult daily dose of glutathione is between 50 mg and 30 g, preferably between 100 mg and 10 g, and more preferably between 200 mg and 3 g once or several times per day.

An interval between doses is not particularly limited. Continuous administration is preferred. Typically, administration is performed for one day to one year, and is preferably performed for one week to three months. Moreover, a pharmaceutical according to the present invention can be used not only on humans, but on other animals (referred to hereafter as non-human animals), as well.

### Embodiments

The present invention is described in further detail with reference to embodiments, below. The technical scope of the present invention, however, is not limited to the embodiments.

Conditions for measurements in the present invention and the embodiments are as follows.

### (Measuring Muscle Tissue pH)

After anesthetic, intermuscular pH of the tibialis anterior muscle and the gastrocnemis muscle was measured using a micro-glass electrode (CMN-141, Chemical Instruments).

### (Measuring PGC-1α Activity)

An amount of PGC-1α in the muscles was measured using a western blot test. Soleus muscle of a sedentary group was homogenized and protein concentration was measured using the BCA method (BCA™ Protein Assay Kit, Thermo SCIENTIFIC). The prepared protein sample was poured into 10% gel (Wako), was separated by electrophoresis, and was transferred to a membrane using a transfer device (iBlot, Invitrogen). After 30 minutes of blocking (EzBlokChemi, ATTO CORPORATION), the sample was cleaned three times for five minutes each in Tris buffered saline-(BIO-RAD Laboratories) Tween 20 (Wako) (TBST), and was reacted with a primary antibody using an anti-PGC-1α antibody (CHEMICON International) for 60 minutes at room temperature. The sample was then cleaned three times for five minutes each in TBST and was reacted with a secondary antibody using anti-rabbit antibody (GE Healthcare) for 60 minutes at room temperature. The sample was then cleaned three times for five minutes each in TBST and was reacted with a coloring agent (ECL plus, GE Healthcare) for four minutes. Using an image analysis device (ImageQuant LAS 4000, GE Healthcare), bands were detected and converted to numerical values. Thereafter, a relative ratio with respect to a control set was calculated.

### (Measuring Mitochondrial DNA)

After homogenizing soleus muscle of the sedentary group with a DNA isolation reagent (DNAzol® BD Reagent, Invitrogen), DNA was extracted. β-actin (nuclear DNA code) and cytochrome oxidase II (COX-II, mitochondrial DNA code) were amplified by a polymerase chain reaction, and an assay of the number of DNA copies was made. An amount of mitochondrial DNA (mtDNA) was calculated using a ratio of the two values.

An effect of glutathione on improvement in exercise function was measured using the following method.

### Experiment 1

### Culture Method and Experimental Protocol

The experiment was conducted using 7-week-old male ICR mice (Shimizu Laboratory Supplies). After one week of preliminary care, the mice were separated into two groups based on body weight: a glutathione group (n=21) and a control group (n=21). Glutathione (KOHJIN Life Sciences Co., Ltd.) was prepared in a 2.0% solution and was administered orally once a day for two weeks to the glutathione group at a dose of 5 μl/g of body weight. The same amount of distilled water was administered orally to the control group for the same period of time. A twelve-hour light/dark cycle was maintained in the enclosure and the mice were able to freely take food and water from a plastic gauge. On the final day, the groups were further divided into a control sedentary group (n=13), a control exercise group (n=8), a glutathione sedentary group (n=13), and a glutathione exercise group (n=8). The exercise groups were given the task of running on a treadmill, after which all groups were dissected.

### Experiment 2

### Exercise and Dissection Protocol

The exercise task employed a treadmill (MK680, Muromachi Kikai). On the day of the exercise task, the sedentary groups were dissected without performing the exercise task, while the exercise groups were given the task of running on a treadmill for 30 minutes at a speed of 25 m/min, then were dissected immediately after completing the exercise task. After measuring the muscle tissue pH while under anesthesia, the soleus muscle of both legs was surgically extracted. The muscles were frozen in dry ice, then maintained at −80° C. until measurement.

### Embodiment 1

### Muscle Tissue pH

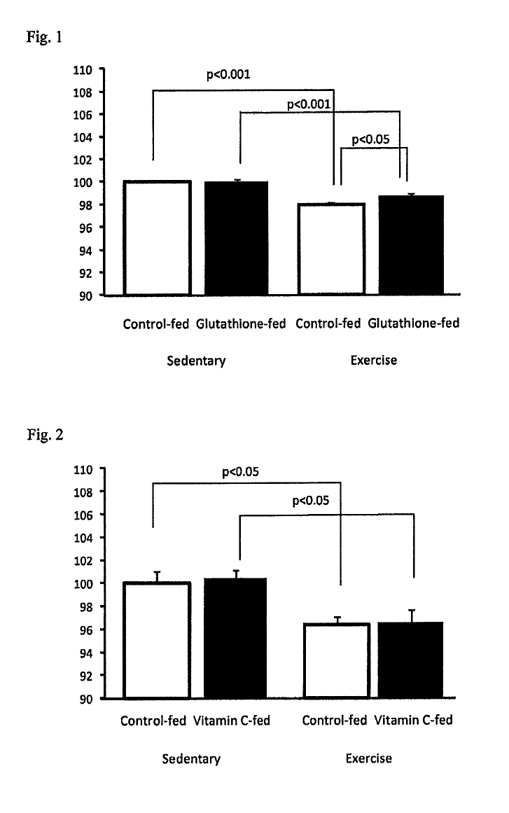

Muscle tissue pH exhibited a significantly lower value after exercise as compared to when sedentary (p<0.001). In addition, the muscle tissue pH exhibited a significantly higher value in the glutathione exercise group as compared to the control exercise group (p<0.05). (See FIGS. 1 and 2.) For comparison, data was obtained in which vitamin C was administered in a manner similar to the glutathione administration.

### Embodiment 2

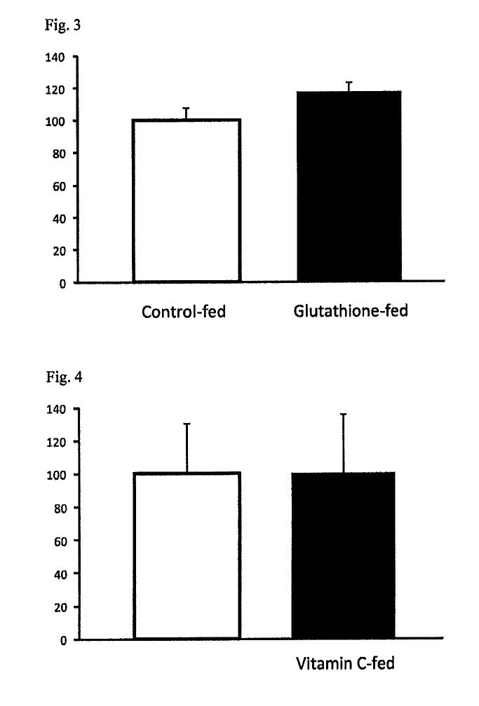

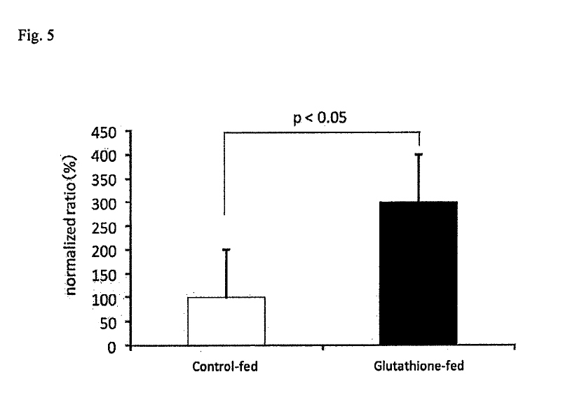

Skeletal Muscle PGC-1α and mtDNA The PGC-1α expression in the soleus muscles exhibited a significantly higher value in the glutathione groups as compared to the control groups (p<0.05). The relative ratio of mtDNA (COX-II/β-actin) exhibited a significantly higher value in the glutathione groups as compared to the control groups (p<0.05). (See FIGS. 3 and 4.) For comparison, data was obtained for PGC-1α expression with vitamin C under conditions similar to the glutathione data.

All of the data was plotted with the mean value±standard error. After performing a two-way analysis of variance in comparing the four groups, a multiple comparison test (Bonferroni) was performed. Student's t-distribution test was performed when comparing two groups. Level of significance was defined at 5%.

The results of the measurements indicated that, in the glutathione-fed groups, no reduction in muscle tissue pH was observed whereas an increase in PGC-1α expression and an increase in mtDNA were observed. Therefore, an improvement in exercise function due to administration of glutathione was confirmed. Meanwhile, the effects of glutathione were not observed with vitamin C.

Given the above, an exercise function enhancer that is safe and contains glutathione as an active component can be provided. In addition, due to activation of PGC-1α, metabolic activity in muscular tissue can be induced and lipid metabolism in the body can be promoted not only while exercising, but when sedentary as well. Therefore, lifestyle diseases such as diabetes can be prevented.

It is noted that the foregoing examples have been provided merely for the purpose of explanation and are in no way to be construed as limiting of the present invention. While the present invention has been described with reference to exemplary embodiments, it is understood that the words which have been used herein are words of description and illustration, rather than words of limitation. Changes may be made, within the purview of the appended claims, as presently stated and as amended, without departing from the scope and spirit of the present invention in its aspects. Although the present invention has been described herein with reference to particular structures, materials and embodiments, the present invention is not intended to be limited to the particulars disclosed herein; rather, the present invention extends to all functionally equivalent structures, methods and uses, such as are within the scope of the appended claims.

The present invention is not limited to the above described embodiments, and various variations and modifications may be possible without departing from the scope of the present invention.

What is claimed is:

1. A method of enhancing exercise function in a subject in need of such treatment, comprising administering an effective amount of glutathione as an active component to enhance exercise function due to an increase in an amount of mitochondrial activity in said subject.

2. A method of enhancing exercise function in a subject in need of such treatment, comprising administering an effective amount of glutathione as an active component to enhance exercise function due to inhibiting reduction in muscle pH.

3. A method of enhancing exercise function in a subject in need of such treatment, comprising administering an effective amount of glutathione as an active component to enhance exercise function due to an increase in an amount of mitochondrial activity and an inhibition of muscle pH reduction.

4. The method of enhancing exercise function of claim 1, comprising administering glutathione as an active component in an amount effective to enhance exercise function further comprising activiation of PGC-1α.

5. The method of enhancing exercise function of claim 1, comprising oral administration of an effective amount of glutathione as an active component.
